(19) United States
(12) Plant Patent Application Publication
(10) Pub. No.: US 2003/0110541 P1
(43) Pub. Date: Jun. 12, 2003

(54) Miniature rose plant named 'Foumich'

(76) Inventors: James Sproul, Bakersfield, CA (US); Betty Jacobs, Bakersfield, CA (US)

Correspondence Address: Sproul Roses By Design, 15800 Strebor Drive, Bakersfield, CA, 93312, US

(21) Appl. No.: 10/013,169

(22) Filed: Dec. 7, 2001

(51) Int. Cl. A01H 5/00

(52) U.S. Cl. PLT/116

(57) ABSTRACT

This invention relates to a new and distinct cultivar of miniature rose, primarily identified by the deep apricot color of its petals and classic hybrid tea form double flowers.

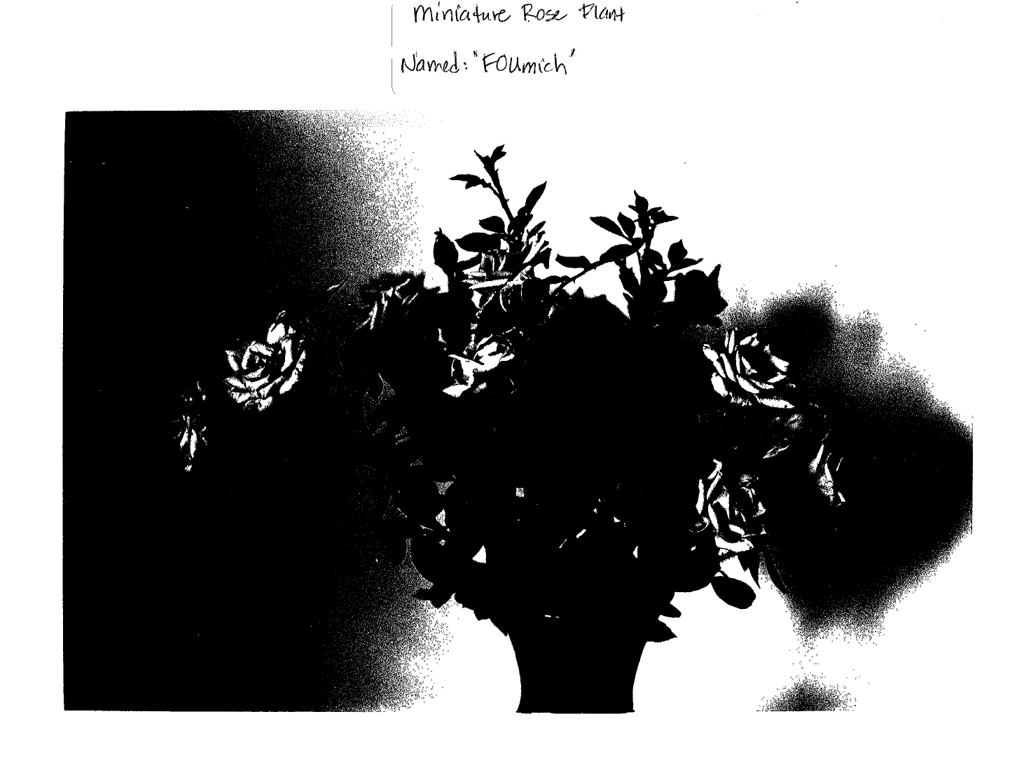

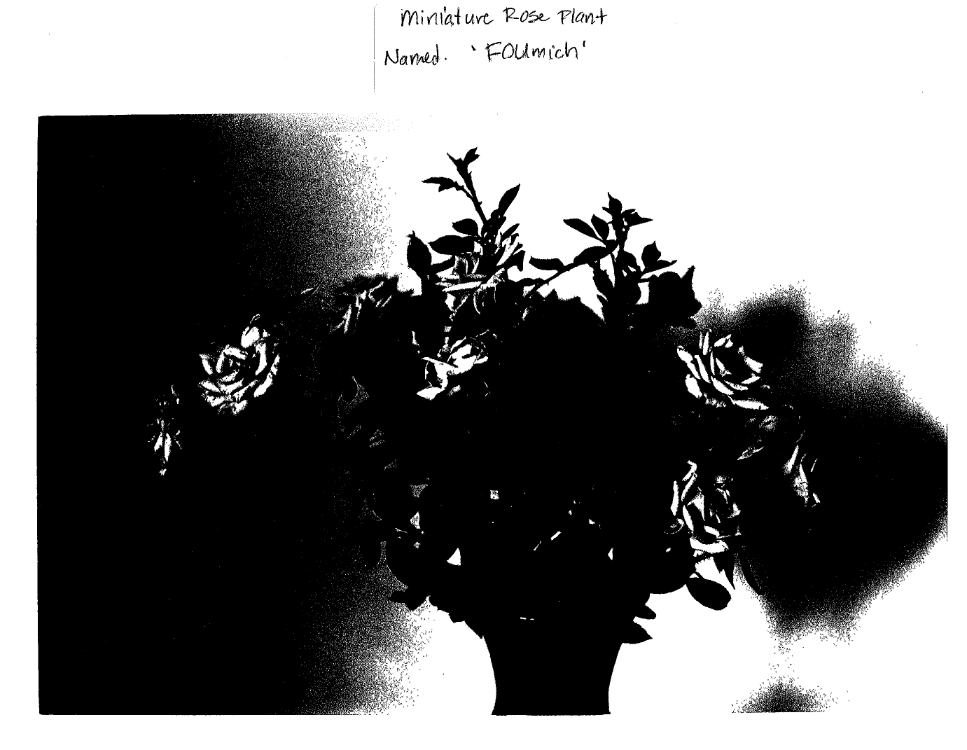

The present invention relates to a new and distinct cultivar of hardy, dwarf, bush type rose plant of the miniature class. The cultivar being primarily characterized—as to novelty by its deep apricot color and classic hybrid tea form double flowers.

The primary objective of this breeding was to produce a new rose variety having the deep apricot flower color, being an intermediate color of the colors of the parent plants, combined with the very good classic formed flowers of the pollen parent ‘FOUsun’, a non-patented miniature rose. The objective was substantially achieved, along with other desirable improvements, as evidenced by the following unique combination of characteristics that are outstanding in the new variety and that distinguish it from its parents, as well as from all other varieties of which I am aware:

1. An early spring bloom, which repeats more rapidly than average and with flowers usually borne singly or in small clusters to the stem.

2. Slight fruity Fragrance.

3. A plant of vigorous, rounded to somewhat spreading shape, with semi-glossy foliage with broad pointed leaflets; the buds and flowers being similar in form to ‘FOUsun’ (‘San Jose Sunshine’, a miniature non-patented rose) but differing in color, being deep apricot instead of yellow blend.

4. As compared to cultivar ‘Prima Donna’, grandiflora rose (U.S. Plant Pat. No. PP5,633); the present cultivar is a miniature rose of more spreading habit and has smaller, apricot colored flowers instead of red-purple or deep fuchsia pink.

5. A plant which grows and blooms satisfactorily both in greenhouse and outdoors; with repeat bloom, is good for growing in pots, and provides excellent garden decoration as well as cut flowers for the home or for exhibition.

The present cultivar of miniature rose was originated by Betty Jacob at her nursery, located at Bakersfield, Calif., under conditions of careful control and observation, as a result of a cross between ‘Prima Donna’, grandiflora rose (U.S. Plant Pat. No. PP5,633) as the seed parent and ‘FOUsun’ (‘San Jose Sunshine’, a miniature rose) as the pollen parent.

Subsequent to origination of the cultivar it was successfully asexually reproduced at the Sproul Roses By Design nursery, located in Bakersfield, Calif. by James Sproul, by budding as well as by cuttings; the reproductions have run true in all respects. The photograph illustrates the cultivar including the foliage, buds, flowers, stems and canes.

The accompanying illustration shows typical specimens of the vegetative growth and flowers of this new variety in different stages of development, depicted in color as nearly true as it is reasonably possible to make the same in a color illustration of this character.

Referring now specifically to the new and distinct cultivar of miniature rose plant, the following is a detailed description thereof in outline; all major color plant identification being by reference to The Royal Horticultural Society (London) colour chart except where common terms of color definition are employed.

Parentage:

Seed parent.—‘Prima Donna’ (U.S. Plant Pat. No. PP5,633).

Pollen parent.—FOUsun (not patented).

Classification:

Botanical.—Rosa hybrida.

Commercial.—Miniature.

Cultivar identification.—‘FOUmich’.

### FLOWER

Flowers borne; Usually singly or in small clusters, on sturdy stems.

Quantity of bloom: Abundant both outside and inside greenhouse.

Bud:

Peduncle.—Medium length; slender but sturdy; erect; medium green, near Yellow Green Group 144C to 144D, sometimes tinted bronze (outdoors); having many fine hairs along its length. Before calyx breaks, size is small to medium.

Form.—Pointed, with few small foliaceous appendages on surface of the bud; with foliaceous parts normally not extending beyond the tip of the bud.

Color.—Medium green, near Green Group 143A, occasionally tinted with bronze (outdoors). As calyx breaks, color is apricot, near Orange Red Group 31A or darker.

Bloom:

Size when fully open.—Medium, averaging about 2¼″.

Petalage.—Double — averaging 25 to 30 or more petals, arranged regularly with a few central small petaloids.

Form.—Full, high centered at first, becoming rounded with petals rolled loosely outward and downward. Fully opened flowers may show stamens (not shown in photo).

Sepals.—Permanent; spear shaped; recurved very early in bloom development, green, near Green Group 143A. There are three normally appendaged sepals. There are two unappendaged sepals with serrated edges.

Behavior.—Opens well in all weather; spent petals of flowers drop off.

Flower longevity (August 2001).—Plant in pot — 4 to 6 days or longer. Cut flowers at living room temperature — 5 to 7 days.

Petals.—Fairly thick; satiny on inside and on outside surface.

Shape.—Outside petals: broad lanceolate (wedge shaped) with very small acute tip. Intermediate: broad lanceolate with nearly round or short acute tip. Inside: lanceolate with acute tip.

Color.—(Newly opening flower from plant grown outside in August, 2001, Bakersfield, Calif., photograph taken August 2001)). Outside petal: Outside surface — deep apricot near Orange Red Group 31B (may be darker at times) blending to a slightly lighter apricot color at the petal base. Inside surface — near Orange Group 24D. Intermediate petal: Outside surface similar to outside petal. Inside surface similar to outside petal. Inner petal: Outside surface similar to intermediate petal. Inside surface similar to intermediate petal. Flower (petal) color fades or lightens as flowers age to near Yellow Orange Group 19D on both outside and inside petal surfaces.

Reproductive organs:

Stamens.—Moderately abundant, arranged regularly about the pistil.

Filaments.—Fairly short, white to yellowish.

Anthers.—Well formed, yellow.

Pollen.—Abundant.

Pistils.—Average number.

Styles.—Long; whitish; bunched.

Ovaries.—All enclosed in calyx.

Hips.—Occasional, round to oval, orange when ripe.

Seed.—Some (few).

Foliage:

Leaves.—Compound of five (sometimes 3 or 7) leaflets; medium small); leathery; semi-glossy.

Leaflets.—Shape: Broad lanceolate; apex acute; base — rounded. Color: Upper surface, medium green (can be as dark as Green Group 135A), while lower surface is lighter near Green Group 137C; new growth is normal (medium) green most often tinted reddish. Rachis: medium strength; upper side smooth; grooved; underside smooth or may have several small thorns (or claws).

Stipules.—Medium to short length; medium width; medium to short length narrow points which turn out usually at about 45.degree. or less. Color similar to leaflets.

Disease resistance.—Somewhat susceptible to blackspot, but moderately resistant to powdery mildew.

Habit.—Medium; bush; spreading; much branched. Plant height: 24 to 30 inches. Plant spread: 30-36 inches.

Growth.—Vigorous; internodes medium.

Canes.—Medium slender; sturdy.

Main stems.—Color: Medium green, near Green Group 143A.

Prickles.—Average, slender, long, usually straight to slightly downward, color: brown to gray.

Hairs.—few or none.

The miniature rose plant and its flowers as heretofore described may vary in slight detail due to climate, soil and cultural conditions under which the cultivar may be grown; the present description being of the cultivar as grown outdoor in August 2001 at Bakersfield, Calif.

1. A new and distinct cultivar of rose plant of the miniature class, of hardy, dwarf, bushy, moderately branched habit substantially as illustrated and described, characterized by buds and flowers, deep apricot, the bud being tapered and pointed in the early stage, with the sepals reflexing well ahead of the opening bloom and, with the open flower being double and rounded with 25 to 30 or more petals; and further characterized by a plant of vigorous, somewhat spreading shape with main stems and shoots of average thorniness, the said plant being average to propagate from soft-wood cuttings with medium, semi-glossly foliage, the flowers being usually borne either singly or in small clusters on medium length stems.
